(19) United States
(12) Patent Application Publication
(10) Pub. No.: US 2006/0229356 A1
(43) Pub. Date: Oct. 12, 2006

(54) Use of indomethacin and derivatives as broad-spectrum antiviral drugs and corresponding pharmaceutical compositions

(75) Inventor: Maria Gabriella Santoro, Avellino (IT)

Correspondence Address: YOUNG & THOMPSON, 745 SOUTH 23RD STREET, 2ND FLOOR, ARLINGTON, VA, 22202, US

(73) Assignee: Universita' Degli Studi Di Roma "Tor Vergata", Rome (IT)

(21) Appl. No.: 10/568,071

(22) PCT Filed: Aug. 11, 2004
(86) PCT No.: PCT/EP04/51773

§ 371 Date: Apr. 5, 2006

(30) Foreign Application Priority Data
Aug. 12, 2003 (IT) RM2003A000394

(51) Int. Cl. A61K 31/405

(52) U.S. Cl. 514/420

(57) ABSTRACT

Methods of treating viral disorders comprising administering indomethacin (INDO) and its derivatives and salts as antiviral drugs.

### FIELD OF THE INVENTION

The present invention relates to the use of Indomethacin and derivatives as broad-spectrum antiviral drugs and, in particular, the use of indomethacin and derivatives in pharmaceutical compositions in combination with other active principles for the treatment of viral diseases.

### BACKGROUND OF THE INVENTION

Indomethacin (INDO) is a known compound (Merck Index 4852) and is part of a class of molecules [α(1-aroil-3-indolil)alcanoic acids] described in U.S. Pat. No. 3,161,654. INDO can be chemically characterized as constituted by an indole and a benzene group, variably substituted, and bound via a carbonyl group (C═O) on the indole nitrogen atom.

During our studies on the role of arachidonic acid metabolites on virus replication we have identified some prostaglandins with potent antiviral activity. We have shown that these molecules have a particularly effective antiviral action in experimental models of infection with parainfluenza virus, influenza virus, herpesvirus and rotavirus (Santoro M G, Trends in Microbiology, 5:276-281, 1997). To determine whether a block of prostaglandin synthesis could result in an enhancement of virus replication, we utilized indomethacin (INDO) as an inhibitor of cyclo-oxygenase (COX), the enzyme that catalizes the main reaction in prostaglandin synthesis. On the basis of our previous findings, these studies were expected to show that INDO would enhance virus replication.

There is a large amount of literature on the effect of INDO on viral infection. The studies available in the literature, whereas generally converge on an effect of INDO as an anti-inflammatory agent coadjuvant during viral infection, report contrasting results on a possible direct antiviral activity of INDO. In some virus models (i.e. poxvirus and coxsackie virus) INDO was found to have no antiviral effect or to worsen the infection (Khatib R. et al., J. Infect. Dis. 162:997-998, 1990; Zavagno G. et al., J. Gen. Virol. 68:593-600, 1987), whereas in others INDO was able to inhibit virus replication. In particular, an antiviral effect of INDO has been previously shown during infection with Vesicular Stomatitis Virus (VSV) (Mukherjee P K et al., Virology 135:345-355, 1984), HIV-1 (Bourinbaiar A S et al., FEBS Lett. 360:85-88, 1995), and some herpesviruses (Tanaka J et al., Virology 163:205-208, 1988). From these results it can be argued that INDO antiviral activity should be tested separately for each virus since the indication known in the art are contradictory.

### SUMMARY OF THE INVENTION

The applicant has now found, and this is an object of the present invention, an unexpected effect of INDO, that consists in stimulating an antiviral protective response in cells when these are attacked by viruses. This protective activity can be obtained in the presence of INDO alone or in combination with other compounds, that were unexpectedly found to have a synergic interaction with INDO. Therefore another object of the invention is the combination of INDO with prostanoids, antiviral drugs and metals, metal salts and derivatives being comprised. Further objects of the invention will be evident from the detailed description of the invention.

### BRIEF DESCRIPTION OF FIGURES

FIG. 1 shows the protective effect of INDO on the cellular damage caused by paramyxovirus (SV) infection. FIG. 1A: 37RC cells non infected (control); FIG. 1B: 37RC cells infected with SV for 24 hours non treated (SV); FIG. 1C: 37RC cells infected with SV for 24 hours and treated with 400 microM INDO (SV+INDO).

FIG. 2 shows the protective effect of INDO on the cellular damage caused by coronavirus (CCoV) infection. FIG. 2A: A-72 cells non infected (control); FIG. 2B: A-72 cells infected with CCoV for 24 hours non treated (CCoV); FIG. 2C: A-72 cells infected with CCoV for 24 hours and treated with 400 microM INDO (CCoV+INDO). In both models the cells treated with INDO are protected from the destructive cytopathic effect caused by the virus and appear similar to the non infected control cells.

### DETAILED DESCRIPTION OF THE INVENTION

In the present invention the word INDO indicates compounds, in particular indomethacin, basically constituted by an indole and a benzene group, both variably substituted, and bound via a carbonyl group (C═O) on the indole nitrogen atom. Derivatives and salts pharmaceutically acceptable are to be considered comprised in the word INDO, being understood that derivatives and salts are those synthesizable by the expert in the field.

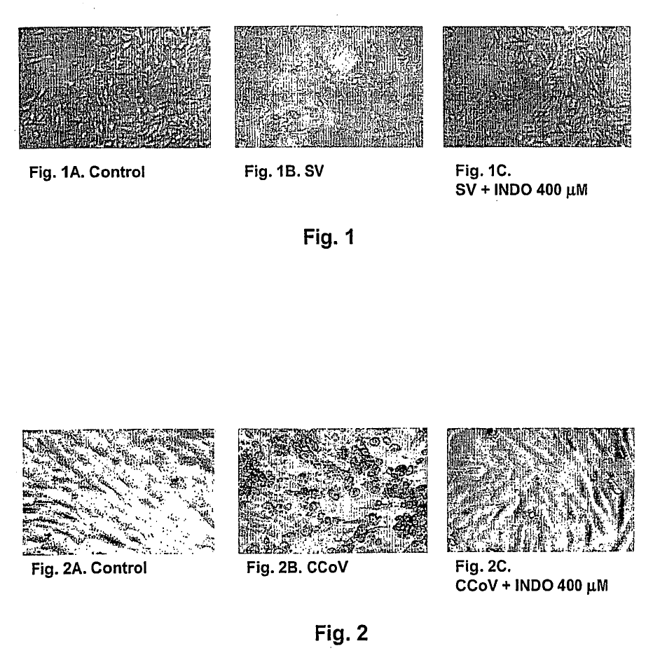

Herein is shown that INDO possesses a broad-spectrum antiviral activity at concentrations non-toxic for the host cell (10-800 microM). An optimal concentration range from 100 to 400 microM was shown to be effective independently from the type of host cell in 5 different types of RNA and DNA virus models. In the case of RNA viruses, as shown in Table 1, a good antiviral activity was shown for single-strand RNA viruses of both negative polarity (parainfluenza Sendai virus and Influenza A WSN virus) and positive polarity (CCoV coronavirus), as well as for double-strand RNA viruses (SA-11 rotavirus). Unexpectedly, antiviral activity against influenza A virus has been shown in human lung cells (Tab. 1). Moreover, INDO treatment is particularly effective against rotavirus and coronaviruses (Tab. 1). The antiviral activity is particularly relevant in the case of coronavirus infection where INDO, at the concentration of 50 microM, inhibits virus production by more than 90% as compared to control. In addition to the antiviral activity, for the first time it has been shown a dramatic and unexpected cytoprotective activity of INDO during infection with paramyxovirus (FIG. 1C) and coronavirus (FIG. 2C). A similar cytoprotective effect has been obtained in a model of rhabdovirus (VSV) infection (data not shown). In the case of DNA viruses the antiviral activity has been confirmed in an experimental model of Herpes Simplex virus type 1 (HSV-1) infection in epithelial HEp-2 cells (Tab. 2).

Surprisingly, INDO antiviral activity is increased, instead of inhibited, by the addition of arachidonic acid metabolites, whose synthesis is known to be blocked by INDO. In particular, combined treatment of INDO with the prostanoid Δ¹²-PGJ₂ is surprisingly effective in the case of influenza virus. In fact, treatment with doses that separately caused a decrease of virus production of approximately 30%, if given in combination caused a reduction of more than 90% in virus yield (Tab. 2). This inhibition lasts up to 48 hours after infection; at this time treatment with INDO alone has lost its effect. Unexpectedly, INDO antiviral activity against influenza virus is increased also by be simultaneous treatment with low doses (from 0.1 to 50 microM) of metals administered as such or as the corresponding salts and derivatives. Preferred metals are zinc, gold, selenium, bismuth and cadmium, and particularly preferred are the corresponding salts and derivatives such as, but not limited to: chloride, sulfate, lactate, citrate, iodate, maleate, thiomaleate, diethil-dithiocarbamate, butyl salicilate, fumarates, succinates, porfirin tetrakis metachloride. Particularly preferred are zinc derivatives selected in the group comprising, but not limited to: ZnCl₂, Zn sulfate, Zn lactate, Zn iodate, Zn diethil-dithiocarbamate, Zn butyl salicilate, Zn porfirin tetrakis metachloride. An example of co-treatment of INDO with ZnCl₂ is shown in Tab. 2. Moreover, surprisingly INDO treatment has a co-operative effect in combination with the antiviral drug ribavirin (Tab. 2).

Similar results were obtained in a model of infection with paramyxovirus, a virus that utilizes replicative mechanisms completely different from the ones utilized by influenza viruses. Also in this case the combined treatment with INDO and the prostanoid 15-deoxy-PGJ₂ at concentrations that separately reduce virus production by 40 and 63% respectively, reduced the virus titer by more than 99% (Tab. 2). Similar results were obtained with the prostanoid PGA₁ (data not shown). Also in this model low concentrations of zinc or its derivatives greatly increased the efficacy of INDO treatment. Furthermore, the antiviral effect of low doses of INDO is significantly increased by co-treatment with alpha-interferon (IFN-α), that, by itself, has no effect against this virus when administered to the cells after virus infection (Tab. 2).

A good synergic effect of the prostanold PGA₁ with low doses of INDO has been shown also in the case of DNA virus infection, as, for example, in the case of infection with HSV-1 (Tab. 2).

In the case of HSV-1, it has to be pointed out a good cooperative effect of metals, as indicated above, and in particular zinc and gold administered as such or derivatives, on the antiviral effect of INDO at low doses. Finally, it also has to be pointed out a good co-operative effect of the anti-herpetic drug acyclovir in combination with INDO at low doses (Tab. 2). The co-operative effect of INDO in the different viral models is expected also for at least a compound selected among, but not limited to: antiviral drugs comprising, in addition to ribavirin and acyclovir, amantadine, rimantadine, influenza virus neuroaminidase inhibitors, iodoxuridine, fosfonacetic acid, 3′-azido-3′ deoxythymidine (AZT), 2′,3′-dideoxycytidine (DDC), viral protease inhibitors and in particular HIV protease inhibitors.

Finally, addition of INDO, even at very high concentrations (5-10 mM), to the virus suspension or during virus adsorption on the host cell has no effect on the replication of any of the viruses indicated above, excluding the possibility of an effect of INDO on the virion itself. Moreover, at the active antiviral concentrations, INDO does not inhibit the activation of the transcription factor NF-κB which is induced by the viral infection, and does not activate the cytoprotective transcription factor HSF (Heat Shock Factor) (data not shown). It has instead been observed the synthesis of the endoplasmic reticulum stress protein GRP78/BiP.

The results reported indicate that INDO is a molecule with broad-spectrum antiviral activity, acting on both RNA and DNA viruses independently from the host cell type. Furthermore, the results obtained show that INDO does not act on the isolated virion, but on the host cell with a mechanism different from the ones utilized by other known broad-spectrum antivirals (ribavirin, interferon, cyclopentenone prostanoids). This is due to the fact that: 1) INDO is not a nucleoside analog as ribavirin; 2) INDO is effective on cells also after virus infection, differently from interferon that acts only if administered to cells before infection; 3) differently from prostanoids, in all models of viral infection that we examined, INDO, at the active antiviral concentrations, does not inhibit the factor NF-κB and does not activate the cytoprotective HSF factor; this indicates that the mechanism of INDO cytoprotective activity during viral infection is distinct from the one utilized by cyclopentenone prostanoids.

The fact that INDO acts with a mechanism different from the other known broad-spectrum antiviral agents gives the advantage of an additive or synergic effect in case of co-treatment. Moreover, in the case of interferon, the fact that INDO, differently from interferon, can function also if administered after infection gives the advantage of a faster response to treatment in the case of combination therapy, as compared to interferon alone.

On the basis of the results obtained from these studies the use of INDO is proposed for treatment of a viral disease selected among, but not limited to: tissue cytoprotection; SARS; gastroenteritis, in particular infective gastroenteritis and gastroenteritis caused by Rotavirus; hemorrhagic fevers, in particular those caused by Filovirus, Bunyavirus, Arenavirus and Flavivirus; respiratory diseases, comprising diseases caused by Coronavirus, Parainfluenza and Influenza viruses, Respiratory Syncytial Virus; virus-caused neoplasias, in particular neoplasias caused by HTLV-1 virus, Epstein-Barr virus, Hepatitis B virus, Hepatitis C virus, Papillomavirus, Adenovirus; viral encephalitis; viral diseases in general, comprising those caused by genetically modified viruses, in particular viral diseases caused by viruses selected in the group comprising, but not restricted to: Herpesvirus, Picornavirus (Rhinovirus, Echovirus, Hepatitis A Virus), Hepatitis B Virus, Hepatitis C virus, Togavirus, Retrovirus (HIV, HTLV-1), Bunyavirus, Arenavirus, Rhabdovirus, Flavivirus, Parainfluenza Viruses, A B and C Influenza Viruses, Respiratory Syncytial Virus, Reovirus, Rotavirus, Coronavirus, Parvovirus, Adenovirus, Papovavirus, Papillomavirus, Poxvirus, Filovirus, Measles and Mumps viruses.

The use of INDO is also proposed for treatment of virus infections that cause diseases in mammals, birds, fish and plants, and then, in general, the use of INDO in veterinary medicine, for aqua-culture and agriculture.

INDO may be provided in any suitable form—i.e. it may be used as such or may be used in the form of a pharmaceutically effective derivative. For example it may be used in the form of a pharmaceutically acceptable salt or hydrate. Pharmaceutically acceptable salts include alkali metal salts (e.g. sodium or potassium salts), alkaline earth metal salts (e.g. calcium or magnesium salts) aluminium salts, zinc salts, ammonium salts (e.g. tetra-alkyl ammonium salts), etc. Inorganic acid addition salts (e.g. hydrochlorides, sulphates, or phosphates) or organic acid addition salts (e.g. citrates, maleates, fumarates, succinates, lactates, propionates or tartrates).

INDO and INDO derivatives may be used as such or in combination with the compounds described above for the preparation of pharmaceutical compositions using the conventional methods utilized in pharmacology.

A medicament will usually be supplied as part of a pharmaceutical composition, which may include one or more pharmaceutically acceptable carriers. This pharmaceutical composition will generally be provided in a sterile form. It may be provided in unit dosage form. It will generally be provided in a sealed container, and can be provided as part of a kit. Such a kit is within the scope of the present invention. It would normally (although not necessarily) include instructions for use. A plurality of unit dosage forms may be provided.

Pharmaceutical compositions within the scope of the present invention may comprise one or more of the following: preserving agents, solubilising agents, stabilising agents, wetting agents, emulsifiers, sweeteners, colourants, odourants, salts (compounds of the present invention may themselves be provided in the form of a pharmaceutically acceptable salt—as explained in greater detail below), buffers, coating agents or antioxidants. They may also contain other therapeutically active agents in addition to INDO as described above.

A pharmaceutical composition within the scope of the present invention may be adapted for administration by any appropriate route, for example by the oral (comprising buccal or sublingual), rectal, nasal, topical (comprising buccal, sublingual or transdermal), vaginal or parenteral (comprising subcutaneous, intramuscular, intravenous or intradermal) routes. Such a composition may be prepared by any method known in the art of pharmacy, for example by admixing one or more active ingredients with a suitable carrier. Pharmaceutical compositions may be designed to pass across the blood brain barrier (BBB). For example, a carrier such as a fatty acid, inositol or cholestrol may be selected that is able to penetrate the BBB.

Formulations according to the invention comprise pills, tablets, capsules, lozenges, solutions, dispersions, suspensions, liposome formulations, miscrospheres, nanospheres, creams and oinments, emulsions and aerosol, sprays and collyria, and can be prepared in retard or controlled-release formulations.

The dosage and delivery system may vary depending upon the nature of the treatment, the age and condition of the individual to be treated, etc. and physicians will ultimately determine appropriate dosages to be used. Moreover, such pharmaceutical compositions may contain INDO in combination with other active molecules or adjuvants, selected in consideration of the nature of the disease to be treated.

Here are reported the following examples, that refer to the tables and figures enclosed, and are given only as an illustration of the present invention but should not be considered as a restriction of the same.

### EXAMPLE 1

Table 1 shows the effect of INDO on the replication of the following viruses: paramyxovirus (Sendai, SV), influenza A virus (WSN strain), rotavirus (SA-11 strain) and coronavirus (CCoV, S-378 strain), in 37RC monkey kidney cells (SV), A549 human lung epithelial cells (WSN), MA104 monkey kidney cells (SA-11) and A-72 canine mammary adenocarcinoma cells (CCoV), respectively. Confluent monolayers of cells grown in RPMI-1640 culture medium (Life Technologies, Inc.) supplemented with 5% FCS (fetal calf serum) and antibiotics were infected with SV or WSN virus (5 HAU/10⁵ cells), or with SA-11 or CCoV virus (5 PFU/cell). After 1 hour at 37° C., the virus inoculum was removed and cells were maintained at 37° C. in RPMI-1640 culture medium containing 2% FCS and different concentrations of INDO diluted in ethanol or of ethanol diluent as a control. Virus titers were determined 24 hours after infection by a standard hemagglutination assay for SV, WSN and SA-11 viruses, and by cytopathic effect 50% assay (CPE_{50%}) for CCoV virus as described in F. Pica et al., Antiviral Res. 20:193, 1993. Virus production is expressed as percent of the virus titer in the control samples. Data shown in Tab. 1 represent the average of different experiments. IC₅₀ is the concentration of INDO in microM that causes a 50% reduction of the virus titer.

### EXAMPLE 2

FIGS. 1 and 2 show the cytoprotective effect of INDO. Cells were infected as described in example 1.

FIG. 1 shows the protective effect of INDO on the cellular damage caused by paramyxovirus (SV) infection. FIG. 1A: 37RC cells non infected (control); FIG. 1B: 37RC cells infected with SV for 24 hours non treated (SV); FIG. 1C: 37RC cells infected with SV for 24 hours and treated with 400 microM INDO (SV+INDO).

FIG. 2 shows the protective effect of INDO on the cellular damage caused by coronavirus (CCoV) infection. FIG. 2A: A-72 cells non infected (control); FIG. 2B: A-72 cells infected with CCoV for 24 hours non treated (CCoV); FIG. 2C: A-72 cells infected with CCoV for 24 hours and treated with 400 microM INDO (CCoV+INDO). In both models the cells treated with INDO are protected from the destructive cytopathic effect caused by the virus and appear similar to the non infected control cells.

### EXAMPLE 3

Table 2 shows the effect of the combined treatment with INDO and antiviral drugs, INDO and Zinc, or INDO and cyclopentenone prostanoids on the replication of the following viruses: paramyxovirus (Sendai, SV), Influenza A virus (WSN strain), Herpes Simplex type 1 virus (HSV-1) in 37RC monkey kidney cells (SV), A549 human lung epithelial cells (WSN) and HEp-2 human laryngeal carcinoma cells, respectively. Confluent monolayers of cells grown in RPMI-1640 culture medium supplemented with 5% FCS and antibiotics were infected with SV or WSN virus (5 HAU/10⁵ cells), or with HSV-1 virus (5 PFU/cell). After 1 hour at 37° C., the virus inoculum was removed and cells were maintained at 37° C. in RPMI-1640 culture medium (Life Technologies, Inc.) containing 2% FCS and different concentrations of INDO (at the indicated doses) by itself or in combination with the antiviral drugs type alpha interferon (IFN-α), ribavirin (RIB), or acyclovir (ACY), or with zinc (ZnCl₂) (Sigma Chemical Co., ST. Louis, Mo., USA), or with the cyclopentenone prostanoids 15deoxy-Δ¹²⁻¹⁴-PGJ₂ (15dx-PGJ₂), Δ¹²-PGJ₂ or PGA₁ (Cayman Chemicals, Ann Arbor, Mich., USA) at the indicated doses. Virus titers were determined 24 hours after infection by a standard hemagglutination assay for SV and WSN viruses, and by CPE_{50%} assay for HSV-1 virus. Virus production is expressed as percent of the virus titer in the control samples. Data shown in Tab. 2 represent the average of different experiments.

TABLE 1
Effect of INDO treatment on the replication of SENDAI virus (SV),
INFLUENZA A VIRUS (WSN), ROTAVIRUS (SA 11) and
CORONAVIRUS (CCoV)

| | INDO μM* | | | | | | |
|---|---|---|---|---|---|---|---|
| | 0 | 25 | 50 | 100 | 400 | 800 | IC₅₀ |
| SV | 100 | 87 | 75 | 60 | 0 | 0 | 130 |
| WSN | 100 | 100 | 100 | 100 | 50 | 7 | 400 |
| SA 11 | 100 | 29 | / | 25 | 0 | 0 | <25 |
| CCoV | 100 | / | 5.8 | 2.8 | 0.2 | <0.03 | <50 |

*Virus yield is expressed as % of control

TABLE 2
Effect of treatment with INDO and antiviral drugs, INDO and Zinc,
or INDO and cyclopentenone prostanoids on the replication of Sendai virus (SV),
Influenza A virus (WSN) and Herpes Simplex virus type 1 (HSV-1)

| | | INDO | IFN | | INDO | ZnCl₂ | | INDO | 15dx-PGJ₂ | |
|---|---|---|---|---|---|---|---|---|---|---|
| | CONTROL* | (100 μM) | (5000 Ul/ml) | TC | (100 μM) | (50 μM) | TC | (100 μM) | (1 μM) | TC |
| SV | 100 | 50 | 100 | 25 | 60 | 60 | 14 | 60 | 37 | 0.8 |
| | | INDO | RIB | | INDO | ZnCl₂ | | INDO | Δ¹²-PGJ₂ | |
| | CONTROL | (400 μM) | (50 μM) | TC | (200 μM) | (50 μM) | TC | (400 μM) | (5 μM) | TC |
| WSN | 100 | 67 | 58 | 33 | 75 | 67 | 8 | 67 | 67 | 6 |
| | | INDO | ACY | | INDO | ZnCl₂ | | INDO | PGA₁ | |
| | CONTROL | (100 μM) | (1 μM) | TC | (25 μM) | (50 μM) | TC | (25 μM) | (25 μM) | TC |
| HSV-1 | 100 | 9.7 | 7.7 | 0.4 | 28.2 | 8.3 | 3.9 | 38.1 | 9 | 0.6 |

*Virus yield is expressed as % of control
Legend:
acyclovir (ACY),
ribavirin (RIB),
Interferon (IFN),
Co-Treatment (TC)

1-53. (canceled)

54. A method of treating SARS (Severe Acute Respiratory Syndrome) treatment, comprising administering an effective amount of INDO and its pharmaceutically acceptable salts and derivatives to a subject in need of said treatment thereof.

55. A method of treating hemorrhagic fevers, comprising administering an effective amount of INDO and its pharmaceutically acceptable salts and derivatives to a subject in need of said treatment thereof.

56. The method according to claim 55 wherein fever is caused by Filovirus, Bunyavirus, Arenavirus or Flavivirus.

57. A method of treating viral diseases caused by viruses selected from the group consisting of: Parainfluenza viruses, A, B and C influenza viruses; Respiratory Syncytial Virus; Reovirus; Rotavirus; Coronavirus; Parvovirus; Adenovirus; Papovavirus; Papillomavirus; Poxvirus; and Filovirus, comprising administering an effective amount of INDO and its pharmaceutically acceptable salts and derivatives to a subject in need of said treatment thereof.

58. The method according to claim 54 wherein INDO salts and derivatives comprise alkali metal salts, alkaline earth metal salts, aluminium salts, zinc salts, ammonium salts, inorganic acid addition salts, and/or organic acid addition salts.

59. The method according to claim 54 wherein said INDO is in the form of a veterinary medicine, agriculture product, or aquaculture product.

60. The method according to claim 54 wherein INDO is administered at concentrations ranging between 10 and 1000 microM.

61. Use according to claim 54, wherein INDO is administered at concentrations ranging between 100 and 400 microM.

62. The method according to claim 54 wherein INDO is administered in combination with pharmaceutically acceptable carriers comprising: preserving agents, solubilising agents, stabilising agents, wetting agents, emulsifiers, sweeteners, colourants, odourants, salts, buffers, coating agents and or antioxidants.

63. The method according to claim 54 wherein INDO is administered by the oral, rectal, nasal, topical, vaginal, parenteral routes.

64. The method according to claim 54 wherein INDO is administered in combination with a carrier that is able to penetrate BBB.

65. A pharmaceutical compositions comprising INDO in combination with pharmaceutically effective amounts of at least one compound comprising: Interferon, metals and corresponding salts and derivatives, prostanoids, antiviral drugs; and corresponding mixtures for the treatment of diseases comprising: tissue cytoprotection, SARS, gastroenteritis, hemorrhagic fevers, respiratory diseases, diseases caused by Coronavirus, Parainfluenza viruses, Influenza viruses, Respiratory Syncytial Virus, virus-caused neoplasias, viral encephalitis, viral diseases in general, and viral diseases caused by genetically modified viruses.

66. The composition according to claim 65 wherein INDO salts and derivatives comprise alkali metal salts, alkaline earth metal salts, aluminium salts, zinc salts, ammonium salts, inorganic acid addition salts, and/or organic acid addition salts.

67. The composition according to claim 65, wherein prostanoids comprise: cyclopentenone prostanoids, PGA₁, PGA₂, PGJ₁, PGJ₂, delta¹²-PGJ₂, and/or 15-deoxy-delta^{12,14}-PGJ₂.

68. The composition according to claim 65, wherein the metals comprise: zinc, gold, selenium, bismuth and cadmium, or corresponding salts and derivatives.

69. The composition according to claim 65, wherein the metal salts and derivatives comprise: chloride, sulfate, lactate, citrate, iodate, maleate, thiomaleate, diethil-dithiocarbamate, butyl salicilate, fumarate, succinate, and/or porfirin tetrakis metachloride.

70. The composition according to claim 65, wherein the metal is Zn and corresponding salts and derivatives comprise: ZnCl₂, Zn sulfate, Zn lactate, Zn iodate, Zn diethil-dithiocarbamate, Zn butyl salicilate, and/or Zn porfirin tetrakis metachloride.

71. The composition according to claim 65, wherein said antiviral drugs comprise: Ribavirin, acyclovir, amantadine, rimantadine, influenza virus neuroaminidase inhibitors, iodoxuridine, fosfonacetic acid, AZT, DDC, and/or viral protease inhibitors.

72. The composition according to claim 71, wherein the viral protease inhibitors are HIV protease inhibitors.

73. The composition according to claim 65 wherein INDO is administered in combination with pharmaceutically acceptable carriers comprising: preserving agents, solubilising agents, stabilising agents, wetting agents, emulsifiers, sweeteners, colourants, odourants, salts, buffers, coating agents, and/or antioxidants.

74. The composition according to claim 65 wherein INDO is in an oral, rectal, nasal, topical, vaginal, or parenteral form.

75. The composition according to claim 65 wherein INDO in combination with a carrier that is able to penetrate BBB.

76. The composition according to claim 65, further comprising a pharmaceutically acceptable vector.
